(19) United States
(12) Patent Application Publication
(10) Pub. No.: US 2007/0060554 A1
(43) Pub. Date: Mar. 15, 2007

(54) Fungal and bacterial infection medication

(76) Inventors: Nathan Kaplan, Cave Creek, AZ (US); Vincent Chiarelli, Cave Creek, AZ (US)

Correspondence Address: US CORPORATIONS, INC., P.O. BOX 234265, ENCINITAS, CA, 92023, US

(21) Appl. No.: 11/227,514

(22) Filed: Sep. 15, 2005

(51) Int. Cl. A61K 31/573; A61K 31/496

(52) U.S. Cl. 514/171; 514/253.08; 514/254.07

(57) ABSTRACT

A fungal and bacterial infection medication and method comprised of Ketoconazole (Ke), Enrofloxacin (Flo) and Diprionate (Dip). Diprionate for the purpose of treating fungal infections and decrease inflammation in animal ear canals.

### CROSS-REFERENCE TO RELATED APPLICATION

### STATEMENT REGARDING FEDERALLY SPONSORED RESEARCH OR DEVELOPMENT

Not applicable

### REFERENCE TO SEQUENCE LISTING

Not applicable

### FIELD OF THE INVENTION

The present invention relates generally to methods of treating fungal infections and inflammation by administration of a combination of bactericidal and anti-inflammatory including Ketoconazole (Ke), Enrofloxacin (Flo) and Diprionate (Dip).

### BACKGROUND OF THE INVENTION

This is a combination of three pharmaceuticals that work to treat fungal and bacterial infections as well as the inflammation that often accompanies infections of this nature. The first pharmaceutical is Ketoconazole (Ke), an imidazole broad spectrum antifungal agent, which impairs the synthesis of ergo sterol, the main sterol of fungal cell membranes, allowing increased permeability and leakage of cellular components. The second pharmaceutical is Enrofloxacin (Flo) which is a synthetic, broad spectrum antibacterial agent that demonstrates a bactericidal activity. The fluorine molecule provides increased potency against gram-negative organisms and broadens the spectrum to include gram-positive organisms. The third pharmaceutical is Betamethasone Diprionate (Dip) which is classified as a corticosteroid and functions to decrease inflammation.

The invention will be the gold standard for treating bacterial as well as fungal infections in the ear canal(s) of canines, felines and other animals if warranted. This will replace products such as otomax-Hc ear drops.

This medication will be utilized by Veterinarians in treating fungal infections of the ear canal in canines, felines and other animals if warranted. The medication will be provided in a 45 ml dropper bottle, which allows placement of 15 drops into the affected ear canal(s) twice daily for 10 days, then once weekly for maintenance as directed.

The present invention discloses a topical antimicrobial and anti-inflammatory pharmaceutical composition and methods of using the same. The topical antimicrobial pharmaceutical composition includes a safe and effective amount of Acetic Acid, Lotrimin and Betamethasone Diprionate.

Further disclosed does a method of treating and preventing microbial related conditions in animals comprise topically applying, to the afflicted areas of animal in need of such treatment, a safe and effective amount of the above composition.

The present invention relates generally to topical pharmaceutical compositions and, more particularly, to primarily aqueous solutions exhibiting unique antimicrobial and/or anti-parasitic properties.

The use of antimicrobial agents plays an important part in current medical therapy. This is particularly true in dermatology, where the most effective course of treatment for skin, mucous membranes, hair lesions, or infections frequently includes the use of a topical antimicrobial agent. The present invention includes topical compositions and methods which provide unique performance against a broad spectrum of microbes with excellent speed.

Various carboxylic acids are known to be good antimicrobial agents. However, when these acidic solutions are directly applied to an animal's skin, they may interfere with wound healing, cause irritation, inflammation or the like. Also, these solutions have poor solubility properties in water which, in turn, limits the efficacy of aqueous solutions employing these materials.

It is also well recognized that there is presently a need for a mild antimicrobial or anti-parasitic which can be safely applied to skin and membranes, such as an animal's ears, that will be substantially harmless to the applied area but which will eliminate, arrest, or substantially reduce the growth of bacteria, fungi, mold and the like. The instant invention meets this need.

Topical antimicrobial and anti-parasite treatment regimens according to the practice of this invention comprise applying a safe and effective amount of the compositions described herein directly to the infected or at-risk skin; particularly; in a veterinary use, on the ear areas or at any other sites particularly susceptible to microbial contamination. The solution may be sprayed, dipped, wiped, dropped, poured, toweled, or the like, onto the area to be treated. Application can be made once, or preferably an amount specified by the veterinary.

The following are disclosed ingredients of the present invention;

Acetic Acid

Acetic acid is the chemical compound responsible for the characteristic odor and sour taste of vinegar. Typically, vinegar is about 4 to 8% acetic acid. As the defining ingredient of vinegar, acetic acid has been produced and used by humans since before the dawn of recorded history. In fact, its name comes from the Latin for vinegar, acetum. Vinegar is formed from dilute solutions of alcohol, such as wine, by the action of certain bacteria in the presence of oxygen. These bacteria require oxygen, and the overall chemical change is the reaction of ethanol with oxygen to form acetic acid and water.

Lotrimin

Lotrimin, also known as Gyne Lotrimin or Clotrimazole, the active ingredient in these medications, is used to treat fungal infections in animals and humans. In preparations for the skin, it is effective against ringworm, athlete's foot, and jock itch. In vaginal creams and tablets, it is used against vaginal yeast infections. In lozenge form, it is prescribed to treat oral yeast infections and to prevent them in people with weak immune systems.

Betamethasone Dipropionate

Betamethasone dipropionate is a synthetic glucocorticoid that is used topically on the skin. (The naturally-occurring glucocorticoid is cortisol or hydrocortisone which is produced by the adrenal gland.) Glucocorticoids have potent anti-inflammatory actions and also suppress the immune response. The augmented formulation (Diprolene) is a highly potent form of betamethasone dipropionate which can suppress the production of hydrocortisone by the body under several circumstances.

Accordingly, it is among the objects of the present invention to provide a novel fungal and bacterial infection medication which is simple to manufacturer, and economical in cost. Another objective of the present invention is to provide a fungal and bacterial infection medication which may be safe and reliable in the practice of veterinary medicine. Still another objective is to provide a fungal and bacterial infection medication which is highly effective when applied in a topical manner to the animal's ear canal.

One object of the present invention is to provide a fungal and bacterial infection medication, which provides substantial effectiveness without adverse and unnecessary side effects.

It is an objective of the invention to provide a fungal and bacterial infection medication which is relatively cost effective and convenient to store.

It is another objective to provide a fungal and bacterial infection medication which is a unitary formula, without elements to be mixed.

It is a further object of the invention to provide a fungal and bacterial infection medication which is designed to be environmentally and medically safe to use.

It is yet a further object of the invention to provide fungal and bacterial infection medication which is inexpensive and easy to manufacture.

In accordance with the above objectives, a fungal and bacterial infection medication provided which comprises the combination of Acetic Acid, Lotrimin, and Betamethasone Diprionate.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the previous following description. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other medication, methods and ingredients for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a fungal and bacterial infection medication and method which has many of the advantages of the closures mentioned heretofore and many novel features that result in a fungal and bacterial infection medication and method which is not anticipated, rendered obvious, suggested, or even implied by any of the prior art tool guides, either alone or in any combination thereof.

It is another object of the present invention to provide a fungal and bacterial infection medication which comprises the combination of Acetic Acid, Lotrimin, and Betamethasone Diprionate.

An even further object of the present invention is to provide a and bacterial infection medication which is susceptible of a low cost of manufacture with regard the combination of all medications and labor, and which accordingly is then susceptible of low prices of sale to the consuming public, thereby making such medication economically available to the buying public.

Still yet another object of the present invention is to provide a bacterial infection medication which provides methods of the prior art some of the advantages thereof, while simultaneously overcoming some of the disadvantages normally associated therewith.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the descriptive matter in which there are illustrated preferred embodiments of the invention.

I have shown but a few embodiments of the invention, it will be apparent to those skilled in the art that the invention may be embodied still otherwise without departing from the spirit and scope of the invention.

Having thus described the invention, what is desired to be secured by a Patent is:

I claim:

1. A fungal and bacterial infection medication and method of treating fungal infections comprising administering a combination of Ketoconazole (Ke), Enrofloxacin (Flo) and Betamethasone Diprionate (Dip) which is classified as a corticosteroid and functions to decrease inflammation.

2. A fungal and bacterial infection medication and method according to claim 1 wherein;
the medication is administered through the veterinary medical practice.

3. A fungal and bacterial infection medication and method according to claim 1 wherein;
the medication is comprised of equal amounts of Ketoconazole (Ke), Enrofloxacin (Flo) and Betamethasone Diprionate (Dip)

4. A fungal and bacterial infection medication and method according to claim 1 wherein;
the physical state of the medication remains as a suspension to penetrate deeper into the ear.

5. A fungal and bacterial infection medication and method according to claim 1 wherein;
the medication is maintained, stored and administered in a single container with the pharmaceutical complex pre-mixed.
